(19) United States
(12) Patent Application Publication
(10) Pub. No.: US 2008/0267878 A1
(43) Pub. Date: Oct. 30, 2008

(54) Targeted Imaging And/Or Therapy Using The [3+2] Azide-Alkyne Cycloaddition

(75) Inventors: Marc Stefan Robillard, Eindhoven (NL); Holger Gruell, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/088,980

(22) PCT Filed: Sep. 29, 2006
(86) PCT No.: PCT/IB2006/053556

§ 371 Date: Apr. 2, 2008

(30) Foreign Application Priority Data
Oct. 4, 2005 (EP) 05109189.0

(51) Int. Cl. A61K 49/00

(52) U.S. Cl. 424/9.1

(57) ABSTRACT

The use of a selective chemical and bioorthogonal reaction providing a covalent ligation such as the [3+2] cycloaddition, in targeted molecular imaging and therapy is presented, more specifically with interesting applications for pre-targeted imaging or therapy. Current pre-targeted imaging is hampered by the fact that it relies solely on natural/biological targeting constructs (biotin/streptavidin). Size considerations and limitations associated with their endogenous nature severely limit the number of applications. The present invention describes how the use of an abiotic, bio-orthogonal reaction which forms a stable adduct under physiological conditions, by way of a small or undetectable bond, can overcome these limitations.

### FIELD OF THE INVENTION

The present invention relates to novel compounds, kits and methods, for use in medical imaging and therapy. The present invention also relates to novel compounds and kits for pre-targeted imaging and/or therapy and to methods of production and use thereof.

### BACKGROUND TO THE INVENTION

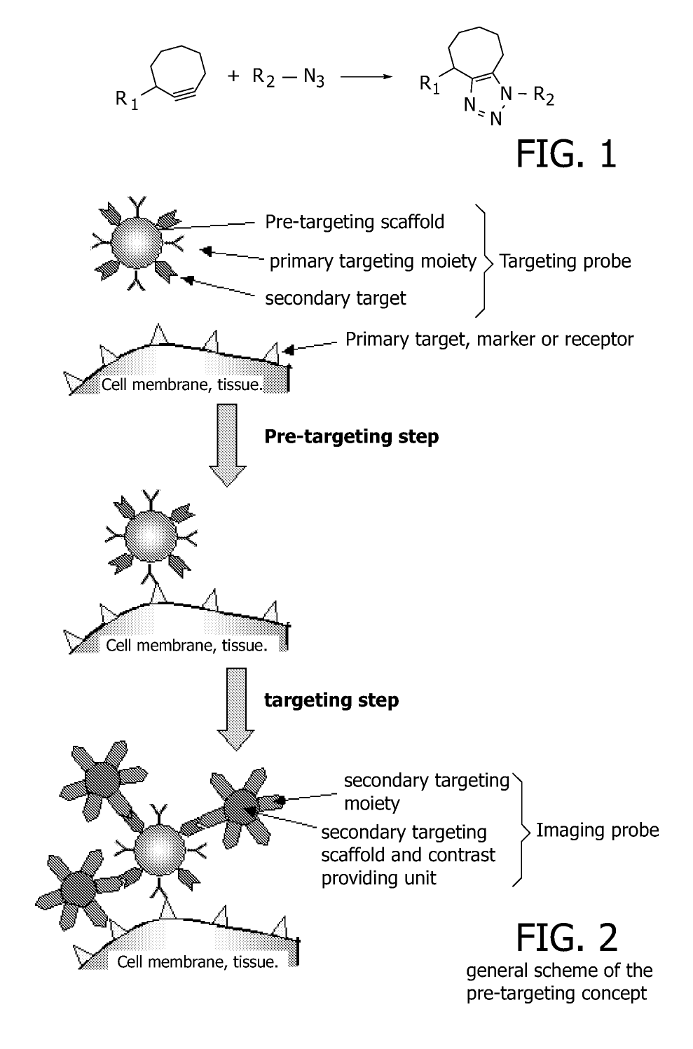

A chemoselective ligation, based on the [3+2] azide-alkyne cycloaddition is known in the art (FIG. 1).

In medical imaging modalities, the use of contrast agents (materials which enhance image contrast, for example between different organs or tissues or between normal and abnormal tissue) is well established. The imaging of specific molecular targets that are associated with disease allows earlier diagnosis and better management of disease. Of particular interest, therefore, are contrast agents that distribute preferentially to distinct body sites, e.g. tumor cells, by virtue of active targeting. Such active targeting is achieved by the direct or indirect conjugation of a contrast-enhancing moiety to a targeting construct. The targeting construct binds to cell surfaces or other surfaces at the target site or is taken up by the cell.

An important criterion for a successful imaging agent for use on living humans and animals is that it exhibits a high target uptake while showing a rapid clearance (through renal and/or hepatobiliary systems) from non-target tissue and from the blood, so that a high contrast between the target and surrounding tissues can be obtained. However, this is often problematic. For example, imaging studies in humans have shown that the maximum concentration of antibody at the tumor site is attainable within 24 h but that several more days are required before the concentration of a labeled antibody in circulation and in non-target tissue decreases to levels low enough for successful imaging to take place. This is in particular a challenge for nuclear probes, because these constantly produce signal by decaying. Consequently, a sufficient signal to background level has to be reached within several half-lives of the tracer. For MRI probes one could wait long enough for the background signal to diminish before imaging. Also activatable probes or “smart probes” exist for MRI approaches; these produce signal only when they interact with a target or enzyme (see U.S. Pat. No. 6,770,261). However, endogenous receptor densities are often too low for sufficient signal accumulation for MRI.

These problems with slow or insufficient accumulation in target tissue, slow clearance from non-target areas and low contrast agent concentration (especially for MRI) have lead to the application of pre-targeting schemes. FIG. 2 shows a typical pre-targeting scheme. In the pre-targeting step, a primary target, such as a receptor of interest, is selectively identified by way of a primary targeting moiety. In order to allow detection after binding, the primary targeting moiety is linked to a secondary target. In a second targeting step, a secondary targeting moiety is administered which will bind to the secondary target. This secondary targeting moiety is itself bound to a contrast providing unit. Typical examples of secondary target/secondary targeting moiety pairs are biotin/streptavidin or antigen/antibody systems.

There are several problems and disadvantages associated with current (pre)targeted imaging. The main issue being that targeting relies solely on natural/biological targeting constructs (i.e. endogenous receptors, biotin/streptavidin). This leads to a range of drawbacks in particular with respect to size and their endogenous nature.

The entities that carry out the highly selective interactions in pre-targeting in particular (biotin-streptavidin, oligonucleotides as secondary targeting moieties), are very large. Due to the size, the pre-targeting concept is so far basically limited to applications within the vascular system. As a result, pre-targeting with peptides and small organic targeting devices as primary ligands, as well as with metabolic imaging, intracellular and brainimaging, have remained out of reach as the size of the secondary targeting moieties precludes the use of small primary ligands. The bulky secondary targeting moieties affect the properties (i.e. transport, elimination, target affinity/interaction) of the pre-targeting construct as well as the imaging probe. Also, the contrast-providing unit of the imaging probe can affect the properties of the secondary targeting moieties (e.g. loss of affinity of biotin conjugate for avidin).

Furthermore, a number of compounds which are used for (pre-)targeting are degraded by the body. Biotin is an endogenous molecule and its conjugates can be cleaved by the serum enzyme biotinidase. Streptavidin conjugates can cause immune reactions. When antisense pre-targeting is used, the oligonucleotides are subject to attack by RNAse and DNAse. Proteins and peptides are also subject to natural decomposition pathways.

The interactions between the respective partners can be further impaired by their non-covalent and dynamic nature. Endogenous biotin competes with biotin conjugates for streptavidin binding. And finally, naturally occurring targets like cell surface receptors are not always present in sufficient amounts to create contrast during imaging.

The technique of pre-targeting has proven very useful for antibody-based imaging, since their pharmacokinetics are usually too slow for imaging applications despite the high selectivity and specificity for their antigens [Sung et al. (1992), Cancer Res. 52, 377-384; Juweid et al. (1992) Cancer Res. 52, 5144-5153]. Although smaller targeting constructs such as antibody fragments, peptides and organic molecules have more appropriate pharmacokinetics, they could profit from a pre-targeting approach as well, since these constructs can still suffer from slow targeting and clearance (i.e. in dense tissues, tissues with low blood flow, or with intracellular imaging) or insufficient accumulation (small lesions, low receptor density, slow growing tumors). Furthermore, accumulation in the clearance pathway (like hepatobiliary or kidney) or other pathways can obscure the tissue of interest.

A recent development in the imaging field is the move towards generalized tracers in which the labeling chemistry remains largely unchanged, but the underlying molecular structure can be easily modified to image a new molecular target. This would afford a reduction in development time/cost for a new imaging agent. Pre-targeting approaches could allow such a generalization to many targets as the contrast-providing group always stays the same for different applications. Consequently, a faster FDA approval of a new molecular imaging application can be expected, as only the pre-targeting group needs FDA approval.

The present invention provides probes and precursors, kits of probes and precursors, methods of producing such probes and precursors, and methods of applying probes and precursors in the context of medical imaging and therapy.

It is an object of the invention to overcome at least one of the above-mentioned drawbacks.

### SUMMARY OF THE INVENTION

In its broadest aspect, the present invention relates to two components which interact with each other to form a stable covalent bio-orthogonal bond. These components are of use in medical imaging and therapy, more particularly in targeted and pretargeted imaging and therapy.

According to a particular embodiment of the invention the covalent bio-orthogonal bond is obtained by the [3+2] cycloaddition of azides and alkynes, and each of the components of the invention comprise a reaction partner for this reaction i.e. an azide or an alkyne group respectively.

A first aspect of the invention relates to the two components, e.g. as present in a kit. The kit of the invention comprises at least one targeting probe, comprising a primary targeting moiety and a secondary target and at least one further probe which is an imaging probe, comprising a secondary targeting moiety and a label. Alternatively, the second component is a therapeutic probe, comprising a secondary targeting moiety and a pharmaceutically active compound. According to the invention one of the targeting probe or the imaging or therapeutic probe comprises, as secondary target and secondary targeting moiety respectively, either at least one azide group and the other probe comprises at least one alkyne group, said alkyne and said azide groups being reaction partners for the [3+2] cycloaddition.

Particular embodiments of the invention relate to targeting probes wherein the primary targeting moieties bind to a component either within or outside the vascular system, or specifically either to a component in the interstitial space or to an intracellular component.

Particular embodiments of suitable primary targeting moieties for use in the kits of the present invention are described herein and include receptor binding peptides and antibodies. A particular embodiment of the present invention relates to the use of small targeting moieties, such as peptides, so as to obtain a cell-permeable targeting probe.

A further aspect of the invention relates to a method for developing targeting probes for use in the context of the present invention. A particular embodiment of this aspect of the invention relates to the production of a targeting probe for targeting a receptor by way of combinatorial chemistry, whereby the azide group is introduced during the synthesis of the compound library. More particularly, the present invention relates to a method of developing a targeting probe with optimal binding affinity for a target and optimal reaction with an imaging or therapeutic probe, which comprises making a compound library of the targeting moiety of said targeting probe, whereby the secondary target is introduced at different sites on said targeting moiety, and screening the so obtained compound library for binding with the target and with an imaging and/or therapeutic probe. Thus the present invention also provides libraries of lead targeting moieties modified with at least one azide group at the same or different amino acids. The invention further provides a library of derivatives or variations of a specific peptide characterized in that the derivatives are modified with an azide group at different amino acid positions in the amino acid chain of said peptide.

Further particular embodiments of the invention relate to a kit of the above-described targeting probes and one or more imaging probes and/or therapeutic probes and the use thereof. Such an imaging probe will comprise, in addition to the secondary targeting moiety which is a reaction partner in the bio-orthogonal reaction of the present invention, a detectable label, particularly a contrast agent used in traditional imaging systems, selected from the group consisting of MRI-imageable agents, spin labels, optical labels, e.g. luminescent, bioluminescent and chemoluminescent labels, FRET-type labels and Raman-type labels, ultrasound-responsive agents, X-ray-responsive agents, radionuclides for SPECT (single photon emission computed tomography) and PET (Positron Emission Tomography), suitable examples of which are known to the skilled person and are provided herein.

A particular embodiment of the present invention relates to the use of small size organic PET and SPECT labels as detectable labels, which provide for cell-permeable imaging probes.

In a further preferred embodiment the invention relates to an imaging probe comprising a secondary targeting moiety and a label characterized in that said imaging probe comprises as secondary targeting moiety at least one azide group or at least one alkyne group, said alkyne or said azide groups being suitable reaction partners for the [3+2] cycloaddition and in that said label is an imaging label.

Further particular embodiments of the invention relate to a kit of the above-described targeting probes and a therapeutic probe, and its use in targeted therapy.

The imaging probe of the present invention can optionally also comprise a therapeutically active compound, which can for instance be a drug or a radioactive isotope. Alternatively, the imaging probe can, in addition to the detectable label comprise a therapeutically active compound.

Another aspect of the invention provides probes particularly suited for medical imaging. Thus the invention provides an imaging probe comprising a secondary targeting moiety and a label whereby the imaging probe comprises as secondary targeting moiety at least one azide group or at least one alkyne group, the alkyne or azide groups being suitable reaction partners for the [3+2] cycloaddition and the label being an imaging label suitable for imaging using classical techniques including nuclear, MRI, X-ray, ultrasound and the like.

A further aspect of the present invention relates to a combined probe for medical imaging and/or therapy comprising a primary targeting moiety and a detectable label or pharmaceutically active compound whereby the targeting moiety is connected to the detectable label via a triazole. In a particular embodiment of this aspect of the invention, the primary targeting moiety is a peptide.

The present invention further relates to a method of in vitro preparing a combined targeting and imaging or therapeutic probe, comprising a primary targeting moiety and a detectable label or a pharmaceutically active agent, comprising the step of reacting an alkyne comprising detectable label with an azide-comprising primary targeting moiety or reacting an azide-comprising detectable label with an alkyne-comprising primary targeting moiety.

The invention further relates to a method of developing a combined probe for medical imaging or therapy with optimal binding affinity for a target and optimal imaging or therapeutic efficiency, which comprises making a compound library of the targeting moiety of the combined probe, whereby the secondary target is introduced at different sites on said targeting moiety, linking the compounds of said library to a label or pharmaceutically active compound and screening the so obtained compound library for binding with the target and/or for therapeutic efficiency. This method is particularly suited for combined probes wherein the primary targeting moiety is a peptide or a protein.

Thus in a particular embodiment the present invention provides kits for targeted medical imaging or therapeutics comprising at least one target metabolic precursor comprising a secondary target and at least one further probe selected from either an imaging probe comprising a secondary targeting moiety and a label or a therapeutic probe comprising a secondary targeting moiety and a pharmaceutically active compound, whereby one of the target metabolic substrate or the imaging or therapeutic probe comprises, as secondary target and secondary targeting moiety respectively, either at least one azide group and the imaging or therapeutic probe comprises at least one alkyne group, the alkyne and the azide groups being reaction partners for the [3+2] cycloaddition.

Particular embodiments of this aspect of the invention relate to the above described kits wherein the target metabolic precursor comprises the at least one azide group and wherein the imaging or therapeutic probe comprises the at least one alkyne group.

Further particular embodiments of the present invention relate to kits comprising a metabolic precursor, which is selected from a group consisting of sugars, amino acids, nucleosides and choline.

Further particular embodiments of the present invention relate to kits comprising a metabolic precursor and an imaging probe, more particularly an imaging probe comprising a detectable label, which is a contrast agent used in traditional imaging systems. Such a detectable label can be but is not limited to a label selected from the group consisting of MRI-imageable agents, spin labels, optical labels, ultrasound-responsive agents, X-ray-responsive agents, radionuclides, and FRET-type dyes.

In a particular embodiment of the present invention, use is made of reporter probes. Such a reporter probe can be the substrate of an enzyme, more particularly an enzyme which is not endogenous to the cell, but has been introduced by way of gene therapy or infection with a foreign agent. Non-endogenous as referring to a gene in a cell or tissue herein is used to indicate that the gene is not naturally present and/or expressed in that cell or tissue. Alternatively, such a reporter probe is a molecule which is introduced into the cell by way of a receptor or a pump, which can be endogenous or introduced into the cell by way of gene therapy or infection with a foreign agent. Alternatively, the reporter probe is a molecule which reacts to certain (changing) conditions within a cell or tissue environment.

The invention thus provides probes and kits for of use in medical imaging and therapy. Moreover the present invention relates to methods of imaging and methods of treatment using the two components of the invention. According to a particular embodiment the present invention provides for probes and kits for use in targeted and pre-targeted imaging and therapy. Accordingly, the present invention relates to methods of imaging cells, tissues, organs, foreign components, by use of at least two components which are partners in the bio-orthogonal [3+2] cycloaddition reaction. The two components being a targeting probe, target metabolic precursor or reporter probe on the one hand and an imaging probe on the other hand. Moreover, the present invention relates to methods of prevention or treatment, targeting cells, tissues, organs, foreign components, by use of at least two components which are partners in the bio-orthogonal [3+2] cycloaddition reaction. The two components being a targeting probe, target metabolic precursor, or reporter probe on the one hand and a therapeutic probe on the other hand. The present invention further relates to methods of manufacture of the tools used in imaging and therapy of the present invention. Further the invention relates to the use of a targeting probe comprising a primary targeting moiety and a secondary target, wherein the targeting probe comprises as said secondary target at least one azide group or at least one alkyne, the alkyne or the azide groups being suitable reaction partners for the [3+2] cycloaddition, as a tool in targeted medical imaging. Moreover the invention relates to this targeting probe in the manufacture of a tool for medical imaging.

### DETAILED DESCRIPTION OF THE INVENTION

FIG. 1 shows the [3+2] cycloaddition between an azide and a cycloalkyne.

FIG. 2 presents a general scheme of the pre-targeting concept.

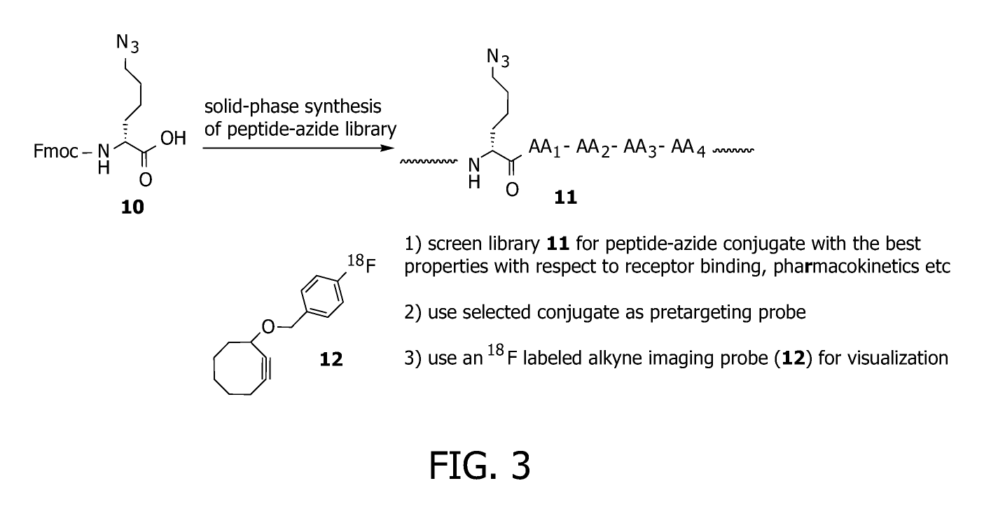

FIG. 3 shows how a binding site for a label or therapeutic compound is included in the design of a combinatorial library for the identification of new leads for a specific target.

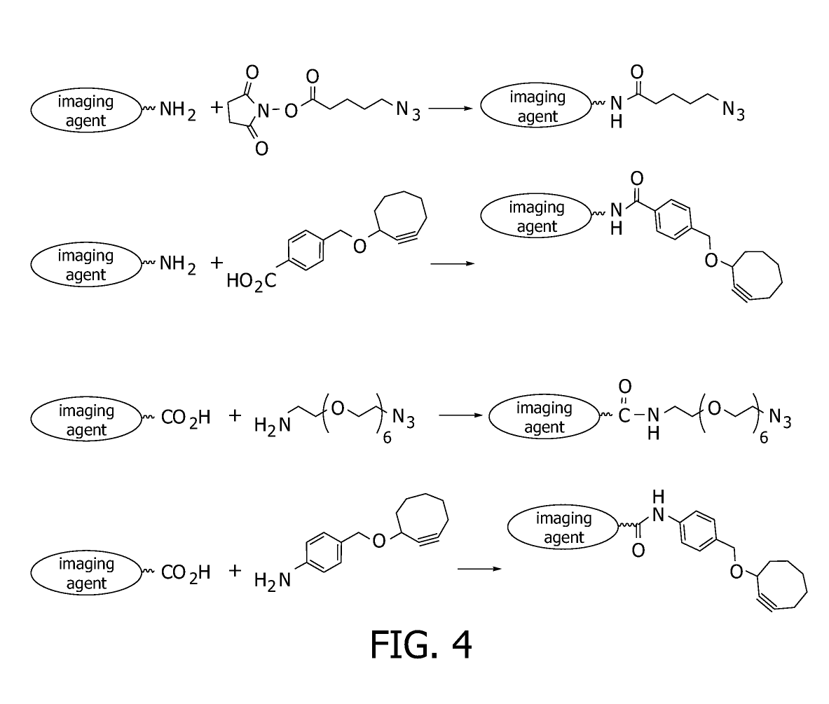

FIG. 4 provides a general synthetic pathway of imaging probes, whereby an imaging agent is conjugated to an azide or cyclooctyne moiety.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

It is furthermore to be noticed that the term “comprising”, used in the description and in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

The present invention provides a solution to the above mentioned limitations of current (pre)targeted imaging, using the [3+2] cycloaddition which is a covalent ligation, especially a biocompatible covalent ligation instead of biologically based interactions, which is a selective chemical and bioorthogonal reaction.

The use of a biocompatible direct covalent reaction between two molecules, which does not occur in nature, solves the drawbacks encountered with recognition mechanisms based on non-covalent reactions in different applications. More particularly, it represents a number of advantages of particular interest in pre-targeting and represents a powerful new tool in molecular imaging.

Embodiments of the present invention provide a chemical reaction wherein the two participating functional groups are much smaller than their biological counterparts in current pretargeting combinations. With the methods of the present invention, two participating functional groups, e.g. azide and alkyne, are used which equal the tremendous selectivity of non-covalent recognition events that occur in many biological processes, such as antibody-antigen binding. In accordance with an aspect of the present invention two participating functional groups are selected that have a finely tuned reactivity so that interference with coexisting functionality is avoided. In accordance with a further aspect of the present invention reactive partners are selected which are abiotic, form a stable adduct under physiological conditions, and recognize only each other while ignoring their cellular/physiological surroundings, i.e. they are bio-orthogonal. The demands on selectivity imposed by a biological environment preclude the use of most other conventional reactions.

Using the method and compounds of the present invention, imaging probes can be rapidly excreted from the body, due to their small size, e.g. through the kidneys, and can provide the desired high tumor accumulation with relatively low non-target accumulation. In nuclear medical imaging the concept of pre-targeting is advantageous, as the time consuming pre-targeting step can be carried out without using radioactive isotopes, while the secondary targeting step using a radioactive isotope, coupled to a small azide or alkyne comprising the secondary targeting moiety, can be carried out faster. The latter allows the use of shorter-lived radionuclides with the advantage, for example, of minimizing the radiation dose to the patient and allowing the usage of PET i.e. Positron Emission Tomography agents instead of SPECT i.e. Single Photon Emission Computerized Tomography agents. In ultrasound imaging the conventional contrast agents have been based on bubbles and have limited contrast agent lifetime. Pre-targeting concepts according to the present invention can circumvent the problem of limited contrast agent lifetime and make the usage of a universal contrast agent possible. Moreover, the present invention is particularly suitable for use in multimodal imaging, optionally using different imaging agents to visualize the same target. Alternatively the imaging probe comprises at least 2 different labels to enable multimodal imaging.

In accordance with a further aspect of the present invention, a pre-targeting approach is used in combination with multidentate ligand systems such as dendrimers, polymers, or liposomes, so that signal amplification, e.g. MRI signals, at target sites can be accomplished.

The application of the [3+2] cycloaddition in molecular imaging opens up pre-targeting to all types and sizes of targeting constructs. This allows intracellular and metabolic imaging to profit from the high target accumulation and low background, attainable through pre-targeting build-up. Likewise, pre-targeted signal amplification schemes, e.g. polyazido and/or polyalkyne dendrimers or liposomes, become available for smaller and more diverse targeting devices. As the reaction partners are abiotic and bio-orthogonal, pre-targeting with the [3+2] cycloaddition is not hampered by endogenous competition and metabolism/decomposition, and affords a stable covalent bond. Choosing a target metabolic pathway, and the corresponding azido-metabolite derivative by virtue of its high flux in, for example, tumor cells compared to normal cells, affords the installation of a high density of artificial azido receptors or other chemical handles on the surfaces of target cells, circumventing the use of endogenous cell surface receptors which can sometimes be at low levels.

Finally, the orthogonal nature of the [3+2] cycloaddition makes it the reaction of choice in the generalization of pre-targeting schemes or conjugation chemistry of imaging agents to a wide range of targeting devices and/or with a great variety of functional groups.

The present invention presents novel applications of the [3+2] cycloaddition as one example of a covalent bonding system that is biocompatible and can be used in the human or animal body. It is believed to be bio-orthogonal.

In the [3+2] cycloaddition, an azide reacts with an alkyne to form a triazole adduct. Especially for cylcoalkynes this reaction can take place without a catalyst such as a Cu catalyst, because of the strain present in the cycloalkyne ring. Also for linear alkynes it is known that the reaction may take place without a catalyst. Reactions between azide and linear alkynes are for example described in Z. Li, T. Seok Seoa, J. Jua, Tetrahedron Letters 45 (2004) 3143-3146.

A “primary target” as used in the present invention relates to a target to be detected by imaging or which is used as the focal point of therapy. For example, a primary target can be any molecule which is present in an organism, tissue or cell. Targets for imaging include cell surface targets, e.g. receptors, glycoproteins; structural proteins, e.g. amyloid plaques; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is upregulated or downreguated in a certain disorder. According to a particular embodiment of the present invention, the primary target is a protein such as a receptor. Alternatively, the primary target may be a compound involved in a metabolic pathway, which is upregulated during a disease, e.g. infection or cancer, such as DNA synthesis, protein synthesis, membrane synthesis and saccharide uptake. In diseased tissues, above-mentioned markers can differ from healthy tissue and offer unique possibilities for early detection, specific diagnosis and therapy especially targeted therapy.

A “targeting probe” as used herein refers to a probe which binds to the primary target. The targeting probe comprises a “primary targeting moiety” and a “secondary target”.

A “primary targeting moiety” as used in the present invention relates to the part of the targeting probe which binds to a primary target. Particular examples of primary targeting moieties are peptides or proteins which bind to a receptor. Other examples of primary targeting moieties are antibodies or fragments thereof which react with a cellular compound. Antibodies can be raised to non-proteinaceous compounds as well as to proteins or peptides. Other primary targeting moieties can be made up of aptamers, oligopeptides, oligonucleotides, oligosacharides, as well as peptoids and organic drug compounds. A primary targeting moiety preferably binds with high specificity, with a high affinity and the bond with the primary target is preferably stable within the body. Other highly suitable primary targeting moieties are polymers. Polymers with multiple azides or alkynes will localize in tumor tissue due to the EPR effect (enhanced permeability and retention). The polymers are too big to extravasate into healthy tissue which leads to selective targeting of tumors.

A “secondary target” as used in the present invention relates to that part of the targeting probe which provides the reaction partner for the [3+2] cycloaddition. In specific embodiments, the secondary target will be one or more azide groups. However, in other particular embodiments, applications are envisaged wherein the secondary target will be one or more alkyne groups.

A “target metabolic precursor” as used herein refers to a substrate of a metabolic pathway which comprises a reaction partner for the covalent cycloaddition, e.g. as secondary target, which according to the present invention reacts with the secondary targeting moiety of the imaging or therapeutic probe described below. The metabolic pathway can be a pathway occurring in each cell (like DNA-, protein- and membrane-synthesis) and can be upregulated during for example cancer or inflammation/infection. Alternatively, the metabolic pathway can be specific for a particular cell type, e.g. cancer cells.

The “imaging probe” comprises a “secondary targeting moiety” and a detectable label, such as for instance a contrast providing unit.

A “secondary targeting moiety” relates to the part of the imaging probe comprising a reaction partner for the [3+2] cycloaddition which reacts with secondary target on the primary targeting probe. In particular embodiments the secondary targeting moiety will comprise the one or more alkyne groups.

A “detectable label” as used herein relates to the part of the imaging probe which allows detection of the probe, e.g. when present in a cell, tissue or organism. One type of detectable label envisaged within the context of the present invention is a contrast providing agent. Different types of detectable labels are envisaged within the context of the present invention and are described herein.

A “therapeutic probe” as used herein refers to a probe comprising a secondary targeting moiety and a pharmaceutically active compound, such as but not limited to a therapeutic compound. Examples of pharmaceutically active compounds are provided herein. A therapeutic probe can optionally also comprise a detectable label.

A “combined probe”, i.e. a “combined targeting and imaging probe” or a “combined targeting and therapeutic probe” or a “combined targeting and imaging and therapeutic probe” as used herein refers to the compound resulting from the binding of the secondary target, e.g. an azide or an alkyne, of the targeting probe with the secondary targeting moiety, e.g. an alkyne or an azide, respectively of the imaging probe. This binding can be in vitro. Thus such a combined probe comprises a primary targeting moiety and a detectable label.

The term “isolated” as used herein refers to a compound being present outside the body or outside a cell or fraction of cell, e.g. cell lysate. With respect to particular features attributed to an isolated probe or combined probe, e.g. a primary targeting probe, imaging probe or a therapeutic probe or a combination thereof, in the context of the present invention, this refers to a probe as present outside the human or animal body, tissue or cell. It does not refer to conjugates which are formed within a body, tissue or cell after the consecutive addition of the constituent components of said conjugate to said body tissue or cell.

In a first aspect the invention relates to a kit for pre-targeting and a kit for targeted medical imaging and/or therapeutics using compounds which react in the [3+2] cycloaddition. In this aspect the invention relates to a kit for targeted medical imaging and/or therapeutics comprising:

at least one targeting probe comprising a primary targeting moiety and a secondary target; and at least one further probe selected from either:

an imaging probe comprising a secondary targeting moiety and a label; or

a therapeutic probe comprising a secondary targeting moiety and a pharmaceutically active compound,

wherein one of the targeting probe or the imaging or therapeutic probe comprises, as secondary target and secondary targeting moiety respectively, either at least one azide group and in that the other probe comprises at least one alkyne group, said alkyne and said azide groups being reaction partners for the [3+2] cycloaddition.

The invention also relates to a method for pretargeting and for targeted medical imaging or therapy wherein this kit is used.

The general concept of pre-targeting is outlined for imaging in FIG. 2. A marker of interest is present on e.g. a cell surface of a certain diseased tissue. This marker is referred to as the “primary target”. In a first pre-targeting step, a targeting probe binds via the primary targeting moiety to the primary target. The targeting probe also carries a secondary target, which will allow specific conjugation to the imaging probe. Optionally, once the targeting probe has reached the primary target and is bound to it, e.g. taking 24 hours to do so, a clearing agent can be used to remove excess targeting probe from the tissue, or organism, if natural clearance is not sufficient. In a second incubation step, e.g. 1-6 hours duration, the imaging probe, which provides the detectable label for the imaging modality, binds to the (pre)-bound targeting probe via its secondary targeting groups.

The advantage of making use of the [3+2] cycloaddition in a pre-targeting strategy is that both alkyne and azide are abiotic and essentially unreactive toward biomolecules inside or on the surfaces of cells and all other regions like serum etc. Thus, the compounds and the method of the invention can be used in a living cell, tissue or organism. Moreover, the azide group is small and can be introduced in biological samples or living organisms without altering the biological size significantly. Using the [3+2] cycloaddition it is possible to bind primary targeting moieties which are large in size, e.g. antibodies, with labels or other molecules using small reaction partners, e.g. azide and alkyne. Even more advantageously, primary targeting moieties can be bound which are relatively small, eg peptides, with labels or other molecules using (matched) small reaction partners, eg azide and alkyne. The size and properties of the targeting probe and imaging probe are not greatly affected by the secondary target and secondary targeting moiety, allowing (pre)targeting schemes to be used for small targeting moieties. Because of this, other tissues can be targeted, i.e. the destination of the probes is not limited to the vascular system and interstitial space, as is the case for current pretargeting with antibody-streptavidin.

According to one embodiment, the invention is used for targeted imaging. According to this embodiment, imaging of specific primary target is achieved by specific binding of the primary targeting moiety of the targeting probe and detection of this binding using detectable labels.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosacharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, VEGF/EGF receptors, matrix metalloproteinase (MMP), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase.

In order to allow specific targeting of the above-listed primary targets, the first targeting moiety of the targeting probe can comprise compounds including but not limited to antibodies, antibody fragments, e.g. Fab2, Fab, scFV, polymers, proteins, peptides, e.g. octreotide and derivatives, VIP, MSH, LHRH, chemotactic peptides, bombesin, elastin, peptide mimetics, carbohydrates, monosacharides, polysaccharides, viruses, drugs, chemotherapeutic agents, receptor agonists and antagonists, cytokines, hormones, steroids. Examples of organic compounds envisaged within the context of the present invention are, or are derived from, estrogens, e.g. estradiol, androgens, progestins, corticosteroids, paclitaxel, etoposide, doxorubricin, methotrexate, folic acid, and cholesterol.

According to a particular embodiment of the present invention, the primary target is a receptor and suitable primary targeting moieties include but are not limited to, the ligand of such a receptor or a part thereof which still binds to the receptor, e.g. a receptor binding peptide in the case of receptor binding protein ligands.

Other examples of primary targeting moieties of protein nature include interferons, e.g. alpha, beta, and gamma interferon, interleukins, and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Alternative examples of primary targeting moieties include DNA, RNA, PNA and LNA which are e.g. complementary to the primary target.

According to a particular embodiment of the invention, small lipophilic primary targeting moieties are used which can bind to an intracellular primary target.

According to a further particular embodiment of the invention, the primary target and primary targeting moiety are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

According to one embodiment, the targeting probe and the imaging probe can be multimeric compounds, comprising a plurality of primary and/or secondary targeting moieties and/or secondary targets. These multimeric compounds can be polymers, dendrimers, liposomes, polymer particles, or other polymeric constructs. Of particular interest for amplifying the signal of detection are targeting probes with more than one secondary target, which allow the binding of several imaging probes.

According to a particular embodiment of the present invention, the compounds and methods of the present invention are used for imaging, especially medical imaging. In order to identify the primary target, use is made of an imaging probe comprising one or more detectable labels. Particular examples of detectable labels of the imaging probe are contrast agents used in traditional imaging systems such as MRI-imageable agents, spin labels, optical labels, ultrasound-responsive agents, X-ray-responsive agents, radionuclides, (bio)luminescent and FRET-type dyes. Exemplary detectable labels envisaged within the context of the present invention include, and are not necessarily limited to, fluorescent molecules, e.g. autofluorescent molecules, molecules that fluoresce upon contact with a reagent, etc., radioactive labels; biotin, e.g., to be detected through binding of biotin by avidin; fluorescent tags, imaging agents for MRI comprising paramagnetic metal, imaging reagents, e.g., those described in U.S. Pat. Nos. 4,741,900 and 5,326,856) and the like.

The radionuclide used for imaging can be, for example, an isotope selected from the group consisting of ³H, ¹¹C, ¹³N, ¹⁵O, ¹⁸F, ⁵¹Cr, ⁵²Fe, ⁵²Mn, ⁵⁵Co, ⁶⁰Cu, ⁶¹Cu, ⁶²Zn, ⁶²Cu, ⁶³Zn, ⁶⁴Cu, ⁶⁶G, ⁶⁷Ga, ⁶⁸Ga, ⁷⁰As, ⁷¹As, ⁷²As, ⁷⁴As, ⁷⁵Se, ⁷⁵Br, ⁷⁶Br, ⁷⁷Br, ^{80m}Br, ^{82m}Br, ⁸²Rb, ⁸⁶Y, ⁸⁸Y, ⁸⁹Sr, ⁸⁹Zr, ⁹⁷Ru, ^{99m}Tc, ¹¹⁰In, ¹¹¹In, ^{113m}In, ^{114m}In, ^{117m}Sn, ¹²⁰I, ¹²²Xe, ¹²³I, ¹²⁴I, ¹²⁵I, ¹⁶⁶Ho, ¹⁶⁷Tm, ¹⁶⁹Yb, ^{193m}Pt, ^{195m}Pt, ²⁰¹Tl, ²⁰³Pb. Other elements and isotopes, such as being used for therapy may also be applied for imaging in certain applications.

The MRI-imageable agent can be a paramagnetic ion or a superparamagnetic particle. The paramagnetic ion can be an element selected from the group consisting of Gd, Fe, Mn, Cr, Co, Ni, Cu, Pr, Nd, Yb, Tb, Dy, Ho, Er, Sm, Eu, Ti, Pa, La, Sc, V, Mo, Ru, Ce, Dy, Tl.

The ultrasound responsive agent can comprise a microbubble, the shell of which consisting of a phospholipid, and/or (biodegradable) polymer, and/or human serum albumin. The microbubble can be filled with fluorinated gasses or liquids.

The X-ray-responsive agents include but are not limited to Iodine, Barium, Barium sulfate, Gastrografin or can comprise a vesicle, liposome or polymer capsule filled with Iodine compounds and/or barium sulfate.

Moreover, detectable labels envisaged within the context of the present invention also include peptides or polypeptides that can be detected by antibody binding, e.g., by binding of a detectable labeled antibody or by detection of bound antibody through a sandwich-type assay.

In one embodiment the detectable labels are small size organic PET and SPECT labels, such as ¹⁸F, ¹¹C or ¹²³I. Due to their small size, organic PET or SPECT labels are ideally suited for monitoring intracellular events as they do not greatly affect the properties of the targeting device in general and its membrane transport in particular. Likewise, the azide moiety is small and can be used as a label for intracellular imaging of proteins, mRNA, signaling pathways etc. An imaging probe comprising a PET label and a (cyclo)alkyne as a secondary targeting moiety is lipophilic and able to passively diffuse in and out of cells until it finds its binding partner. Moreover, both components do not preclude crossing of the blood brain barrier and thus allow imaging of regions in the brain.

According to another embodiment the compounds and methods of the invention are used for targeted therapy. This is achieved by making use of a therapeutic probe which comprises a secondary targeting moiety and one or more pharmaceutically active agents (i.e. a drug or a radioactive isotope for radiation therapy). Suitable drugs for use in the context of targeted drug delivery are known in the art. Optionally, the therapeutic probe can also comprise a detectable label, such as one or more imaging agents. A radionuclide used for therapy can be an isotope selected from the group consisting of ²⁴Na, ³²P, ³³P, ⁴⁷Sc, ⁵⁹Fe, ⁶⁷Cu, ⁷⁶A ⁷⁷As, ⁸⁰Br, ⁸²Br, ⁸⁹Sr, ⁹⁰Nb, ⁹⁰Y, ¹⁰³Ru, ¹⁰⁵Rh, ¹⁰⁹Pd, ¹¹¹Ag, ¹²¹Sn, ¹²⁷Te, ¹³¹I, ¹⁴⁰La, ¹⁴¹Ce, ¹⁴²Pr, ¹⁴³Pr, ¹⁴⁴Pr, ¹⁴⁹Pm, ¹⁴⁹Tb, ¹⁵¹Pm, ¹⁵³Sm, ¹⁵⁹Gd, ¹⁶¹Tb, ¹⁶⁵Dy, ¹⁶⁶Dy, ¹⁶⁶Ho, ¹⁶⁹Er, ¹⁷²Tm, ¹⁷⁵Yb, ¹⁷⁷Lu, ¹⁸⁶Re, ¹⁸⁸Re, ¹⁹⁸Au, ¹⁹⁹Au, ²¹¹At, ²¹¹Bi, ²¹²Bi, ²¹²Pb, ²¹³Bi, ²¹⁴Bi, ²²³Ra, ²²⁵Ac.

Alternatively the drug in the therapeutic probe is selected from sensitizers for photodynamic therapy.

A number of therapeutic compounds presently known already contain an azide, and thus represent useful therapeutic probes which can be used in combination with a targeting probe, for targeted therapy to improve their efficiency. For instance azide-dye conjugates for photodynamic cancer therapy (see WO 03/003806) can be more efficiently targeted to diseased tissue using a pre-targeting strategy.

In yet another embodiment of the present invention, the use of a targeting probe is replaced by selectively incorporating the secondary target of the invention into a target cell or tissue. This is achieved by using metabolic precursor molecules, comprising a secondary target, e.g. an azide reaction partner, that can be incorporated into biomolecules by the metabolism of the cell. The metabolic pathways targeted in this way can be pathways that are common to all cells, such as DNA-, protein- and membrane synthesis. Optionally, these are metabolic pathways which are upregulated in disease conditions such as cancer or inflammation/infection. Alternatively, the targeted metabolic pathways are specific for a particular type of cell or tissue. The target metabolic precursors which can be used in the context of the present invention, include metabolic precursor molecules such as, but not limited to amino acids and nucleic acids, amino sugars, lipids, fatty acids and choline. Imaging of these compounds, such as amino acids, can reflect differences in amino acid uptake and/or in protein synthesis. A variety of sugars can be used for the labelling of carbohydrate structure. Fatty acids can be used for the labelling of lipids in e.g. cellular membranes.

Moreover, a number of analogs of metabolic precursors are known in the art, which can provide particular advantages for use in the context of the present invention. A non-limiting list of examples of metabolic pathways and corresponding metabolic precursors which can be labelled with azide or alkyne are provided below. Some of these become temporarily accumulated into the cell, while others are incorporated into biological macromolecules.

Thus in a further embodiment, the invention relates to a kit for targeted medical imaging and/or therapeutics comprising:

at least one target metabolic precursor comprising a secondary target; and at least one further probe selected from either:

an imaging probe comprising a secondary targeting moiety and a label; or

a therapeutic probe comprising a secondary targeting moiety and a pharmaceutically active compound,

wherein one of the target metabolic substrate or the imaging or therapeutic probe comprises, as secondary target and secondary targeting moiety respectively, either at least one azide group and in that the other probe comprises at least one alkyne group, said alkyne and said azide groups being reaction partners for the [3+2] cycloaddition.

A particular embodiment of the invention relates to the use of reporter probes, i.e. molecules which by their involvement in a cellular process, allow the visualization of a process or cell-type. Such a probe can make use of an endogenous mechanism of the cell, e.g. an endogenous enzyme for which a substrate is provided. Alternatively, such a probe functions by virtue of a foreign gene, referred to as a reporter gene. The reporter gene product can be an enzyme that converts a reporter probe to a metabolite that is selectively trapped within the cell. Alternatively, the reporter gene can encode a receptor or transporter or pump, which results in accumulation of the probe into the cells.

Thus in a further embodiment the invention relates to a kit for targeted medical imaging and/or therapeutics comprising:

at least one reporter probe comprising a secondary target; and at least one further probe selected from either:

an imaging probe comprising a secondary targeting moiety and a label; or

a therapeutic probe comprising a secondary targeting moiety and a pharmaceutically active compound,

wherein one of the reporter or the imaging or therapeutic probe comprises, as secondary target and secondary targeting moiety respectively, either at least one azide group and in that the other probe comprises at least one cycloalkyne group, said alkyne and said azide groups being reaction partners for the [3+2] cycloaddition.

According to the present invention either the targeting probe or the target metabolic precursor molecule on the one hand or the imaging probe or therapeutic probe on the other hand, can include an alkyne or an azide group, as the secondary target or the secondary targeting moiety, respectively, which allow the binding of these probes by the [3+2] cycloaddition.

Especially preferred alkynes are cycloalkynes, especially those which have sufficient ring strain to lead to a reaction with azide which takes place without the need for a catalyst. Linear alkynes that are activated by electron withdrawing groups are also among the preferred alkynes. Especially suitable cycloalkynes are those selected from the group comprising at least 6 carbon atoms, more preferred at least 8 carbon atoms. In a more preferred embodiment, the cycloalkyne is selected from the group comprising cyclooctyne, cyclononyne and cyclodecyne, and derivatives thereof. Cyclooctyne is the most preferred alkyne for use in the current invention. Optionally the cycloalkyne is substituted with electron withdrawing groups This was found to increase the rate of the cycloaddition reaction with azides.

Molecules comprising an azide and suitable for use in the present invention, as well as methods for producing azide-comprising molecules suitable for use in the present invention are known in the art.

A general scheme of synthetic pathways for the production of imaging probes whereby an imaging agent comprising an amine or carboxylic acid is linked to a cycloalkyne or azide moiety is provided in FIG. 4. A similar synthetic pathway is applicable for the production of targeting probes, therapeutic probes, or target metabolic precursors starting from appropriate targeting moieties, pharmaceutical compounds or metabolic precursors respectively, bearing an amine or carboxylic group.

According to another embodiment of the invention, a therapeutic probe is used in combination with an imaging probe. In this embodiment, the therapeutic probe, comprising a therapeutic compound and a secondary target is administered directly, e.g. without a targeting probe, and the secondary targeting moiety is used for detection with an imaging probe. Thus, according to this embodiment, the secondary targeting moiety of the therapeutic probe, which in fact functions as a secondary target, and the secondary targeting moiety of the imaging probe are partners in the [3+2] cycloaddition. This embodiment is of use in therapy planning and monitoring, for instance in AZT (Azidothymidine) therapy, AZT (1) is an anti-retroviral drug and the first antiviral treatment to be approved for use against HIV. It already has an azide installed on the sugar moiety. This azide can be used as a handle to bind a labeled cycloalkyne probe, allowing AZT imaging in a patient.

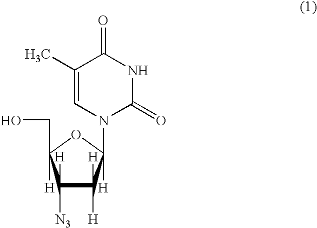

The azide- or alkyne-comprising targeting, imaging and therapeutic probes of the present invention are biocompatible and can be administered in an identical or similar way as conventional molecules which are currently used in medical imaging or therapy. In addition, the detectable labels are known to the skilled person and require conventional methodology and apparatus.

According to a particular embodiment of the invention, the compounds and methods described herein are used in vivo for the imaging or detection of tissues or cell types in the animal or human body. Alternatively, they can be equally used in vitro for the examination of biopsies or other body samples or for the examination of tissues which have been removed after surgery.

As described herein, according to a particular embodiment of the present invention, the targeting probe and imaging or therapeutic probe are provided sequentially, allowing the binding of the targeting probe to its primary target and optionally removal of the excess targeting probe before providing the imaging probe or therapeutic probe. This ensures a higher signal to noise ratio in the image and/or a higher efficiency of the therapeutic and is generally referred to as ‘pre-targeting’ or ‘two-step’ targeting. The compounds of the present invention allow a two-step targeting method wherein the problems (excessive long diffusion to the target and clearance from the organism, decay of imaging compound) traditionally related to the size of the secondary target and secondary targeting moieties (ensuring the recognition and binding between the two steps) are circumvented. Moreover such a ‘two-step’ targeting allows the development of ‘universal’ imaging probes, which can be used in combination with the ‘detection probe’ of interest.

According to a further embodiment the methods and compounds of the present invention are used for targeted signal amplification and/or polyvalency installation. Herein the primary targeting moiety of the targeting probe is conjugated to a dendrimer, polymer or liposome containing multiple alkyne moieties. After receptor binding of the targeting probe through its primary targeting moiety, an imaging probe comprising an azide conjugated to one or more MRI contrast agents, e.g. Gd chelates, or to an ultrasound reporter, e.g. microbubble, is injected. Herein said contrast agents or microbubbles comprise/are functionalized with secondary targeting moieties. The subsequent [3+2] cycloaddition results in a high concentration of MRI contrast agent at the target tissue. Furthermore, the polyvalency at the target site will increase the reaction kinetics with the azide reporter conjugate (imaging probe), affording an efficient target accumulation of MRI contrast agent or microbubbles.

Alternatively, the azide can also be comprised in the targeting probe as mentioned above and the alkyne conjugated to the reporter in the imaging probe.

According to another aspect the methods, compounds of the invention are used without primary targeting probe, but incorporating the secondary target into a precursor molecule to be incorporated into biomolecules by the metabolism of the cell. In this way, general metabolic pathways can be targeted. The above-described alkynes or azides are linked e.g. to sugars, amino acids or nucleosides, which can then be administered to the cell or organism and are incorporated into biomolecules and/or trapped in the cell by the normal metabolism. In a particular embodiment of this aspect of the invention a metabolic pathway, which is upregulated during a disease, like infection/inflammation or cancer, is targeted. Components which can be upregulated in disease conditions include for example DNA, protein, membrane synthesis and saccharide uptake. Suitable building blocks to label these elements include azide-labeled amino acids, sugars, nucleobases and choline and acetate. These azide labeled building blocks are funtionally analogous to the currently used metabolic tracers (also called building blocks) [¹¹C]-methionine, [¹⁸F]-fluorodeoxyglucose (FDG), deoxy-[¹⁸F]-fluorothymidine (FLT), [¹¹C]-acetate and [¹¹C]-choline. Cells with a high metabolism have a higher uptake of these building blocks. Azide-derivatives can enter these pathways and accumulate in and/or on cells. After sufficient build-up and clearance of free building block, an imaging probe, e.g. a probe comprising a radioactive label and a (cell permeable) cycloalkyne as a secondary targeting moiety, is sent in to bind the accumulated azide metabolite. The advantage over normal FDG-type imaging is that there is ample time to allow high build up of the targeting probe before radioactivity is allowed to bind, thus increasing the signal to noise ratio. Alternatively, a metabolic pathway and/or metabolite that is specific for a disease could be targeted.

Yet another aspect of the invention relates to the provision of a combined targeting and imaging or therapeutic probe, for use in imaging and therapy. Thus, according to this aspect the secondary target of the targeting probe and the secondary targeting moiety of the imaging probe or therapeutic probe are allowed to react in vitro, before administration to the cells, tissue or organism. When linking the targeting probe and the imaging or therapeutic probe of the present invention using the [3+2] cycloaddition, there is hardly any increase in size, which is a typical feature of the chemical reaction itself. As a result, the combined targeting probe and imaging probe can be small enough to allow a quick diffusion to the target and a quick clearance from the body. Thus, the [3+2] cycloaddition is envisioned as an orthogonal and general route for the conjugation of imaging agents to targeting constructs. In general, combinations of all of the above-mentioned primary targeting moieties and detectable labels can be produced in vitro and used for this application. It will be understood that for optimal use in vivo, the combined size of the primary targeting moiety and the label should allow sufficiently quick diffusion to the primary target and clearance from the body.

According to a particular embodiment of the above-described aspect of the invention, the combined targeting and imaging/therapeutic probe comprises, as a primary targeting moiety, a peptide which interacts with another protein such as a receptor.

According to another particular embodiment of the above-described aspect of the invention, the detectable label is selected from the group of an organic PET labelled prosthetic group, a metal complex for PET, SPECT, or MRI, a microbubble for ultrasound imaging, and an iodine or barium-containing molecule or vesicle.

An important advantage in the production of the combined targeting and imaging or therapeutic probe of the present invention, is that the individual reagents of the [3+2] cycloaddition (i.e. the targeting probe comprising the secondary target and the imaging or therapeutic probe comprising the secondary targeting moiety) are stable and that the reaction between them occurs in water.

Thus, imaging probes comprising a pendant azide moiety and the targeting probes comprising an alkyne derivative, or vice versa can be produced and stored separately and combined either beforehand as part of production or just before use by the end-user. The reaction is rapid and gives high yields, and the combined product does not require elaborate purification.

Thus, the present invention also envisages combined targeting and imaging or therapeutic probes, or a kit comprising one or more targeting probes for combining with one or more imaging or therapeutic probes. According to a particular embodiment the two components are reacted in vitro, for example just before the imaging procedure and the combined probe is used as such. According to an embodiment, the combined probe is characterized by the presence of triazole adduct which is the result of the [3+2] cycloaddition. [3+2] cycloaddition kit applications are especially envisaged for nuclear imaging agents.

Another aspect of the invention relates to the production of suitable targeting probes for use in the context of the present invention using combinatorial peptide synthesis. In case of peptides, labeling with an azide or alkyne group can disturb the receptor affinity properties. Consequently, after labeling of the targeting moiety, additional time-consuming optimization rounds may be necessary to ensure that the targeting probe has the desired pharmacological properties and receptor affinity. According to this aspect of the present invention, the azide or alkyne is included in the design of a combinatorial library for the identification of new leads for a specific target. The leads generated by such a methodology do not require additional modification but can be directly used as targeting probes in the context of the present invention. According to a particular embodiment, an amino acid building block carrying an azide residue (10) is incorporated at any desired position in the peptide chain during combinatorial preparation of a peptide library (11) (FIG. 3). Peptide-azide-label or peptide-azide-therapeutic conjugates with optimal receptor binding affinity can then be identified for use in targeted imaging or targeted therapeutics, respectively. The combinatorial library thus obtained can comprise an array of a prior identified lead molecule in which the amino acid building block carrying an azide residue is introduced in different positions, to identify by a screening the molecule which displays minimal interaction of the label/therapeutic with the binding of the lead to its target.

Another aspect of the invention relates to the use of the [3+2] cycloaddition in the preparation of targeted imaging or therapeutic agents, corresponding to the combined probes described herein.

Generally, targeted imaging agents are developed by labeling a known targeting group, such as a receptor-binding moiety, e.g. a bioactive peptide or an organic drug-like structure, with a label, such as a radioactive isotope, a metal chelate or an organic fluorophore. The nuclear isotopes are usually bound via a chelate group, e.g. SPECT agents, or an aromatic prosthetic group for halogen labeling. Similarly, targeted therapeutic compounds are developed by linking a known targeting group to a therapeutic compound. These added labeling groups or therapeutic compounds can significantly alter the properties and receptor affinity of the targeting moiety, leading to a sub-optimal targeted imaging agent or therapeutic. Consequently, for each labeled targeting moiety, additional time-consuming optimization rounds of the conjugate with respect to pharmacological properties and target affinity are necessary. According to the present invention, the [3+2] cycloaddition can be used to facilitate the development of such targeted imaging or therapeutic agents. Selective incorporation of the azide or alkyne at different positions is included in the synthesis of the peptide acting as targeting moiety. This optimization can be applied for the development of targeted probes or therapeutics based on known peptide moieties, i.e. incorporating the azide or alkyne at different positions in the known peptide sequence. Alternatively, this can be applied in the development of new targeting moieties, e.g. by inclusion in the design of the combinatorial library of new leads for a specific target. The combinatorial library is then bound to the desired label or therapeutic using the cycloaddition and screened for both optimal binding affinity to the target and label/therapeutic efficiency.

Optionally, an alkyne linked to a cold isotope, e.g. non-radioactive F, I, etc, is coupled to all azide-functionalized library members for studying binding affinities. The “hot” analog can then be obtained by coupling of the azide-peptide to the alkyne labeled with the corresponding hot isotope.

The leads generated by such a methodology do not require additional modification but can, after binding of the label or therapeutic compound to the provided binding site, be directly applied as imaging and/or therapeutic agents.

The probes and kits of the present invention are of use in medical imaging and therapy, more particularly ‘targeted’ imaging and therapy. The term ‘targeted’ relates to the fact that the imaging label or pharmaceutically active compound upon administration to the patient specifically interacts with or is introduced into a target molecule. This can be achieved according to the present invention by use of a targeting probe comprising a targeting moiety or by use of a target metabolic substrate. Alternatively this can be obtained by providing a combined targeting and imaging or therapeutic probe (i.e. administration of the two components of the present invention as a combined probe). This target molecule can be specific for a particular type of cell or tissue or can be common to all cells or tissues in the body. A particular aspect of the present invention relates to ‘pretargeted’ imaging or therapy. This aspect requires the separate use of the two components of the present invention and relates to the separation in time of the administration to the patient of the component which comprises the targeting moiety or ensures the targeting by being a substrate of a particular reaction and the component which ensures the image or therapeutic effect. The time in between administration of the two components can vary but ranges from about 10 minutes to several hours or even days.

Optionally the primary targeting moiety or building block already comprises a detectable label. Preferably this label is different from the label that is introduced in a next step in the [3+2] cycloaddition. Administration of the building block or primary targeting moiety with label such as FDG functionalised with azide, gives rise to an FDG like image, which may in a second step be overlayed with the image that is obtained from the activation step with a labeled alkyne. This combination of two imaging labels, one being present in the building block or primary targeting moiety and the other linked to the alkyne that is administered thereafter, has as potential advantages better target localization, artifact elimination, delineation of non relevant clearance and other pharmacokinetic pathways.

The probes of the invention can be administered via different routes including intravenous injection, oral administration, rectal administration and inhalation. Formulations suitable for these different types of administrations are known to the skilled person.

Therapeutic probes or imaging probes comprising a pharmaceutical composition according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include saline, buffered saline, dextrose, water, glycerol, ethanol, and combinations thereof. The formulation should suit the mode of administration.

The invention is illustrated by the following non-limiting examples.

### EXAMPLES

### Example 1

### Pre-Targeted Imaging of Neuroendocrine Tumours

A targeting probe comprising a somatostatin receptor-binding peptide; e.g. representing a primary targeting moiety in accordance with FIG. 2, linked to an azide group, e.g. as a secondary target, is injected into a subject. After binding of the targeting probe to the primary target, e.g. the somatostatin receptor, present for example in high concentration on neuroendocrine tumours, and clearance of unbound targeting probe, an imaging probe comprising a ¹⁸F-label, i.e. radioactive linked to a cyclooctyne group, which acts as secondary targeting moiety, is injected into the subject, e.g. animal or human; where it binds the immobilized azide. The presence of the neuroendocrine tumor can thus be visualised by the radioactive isotope providing the contrast. Alternatively, the secondary targeting moiety of the imaging probe contains the azide while the cyclooctyne is the secondary target in the targeting probe. Cyclooctyne—(2) or azide-labeled (3) amino acids can be incorporated in a receptor-binding peptide for the production of the targeting probe.

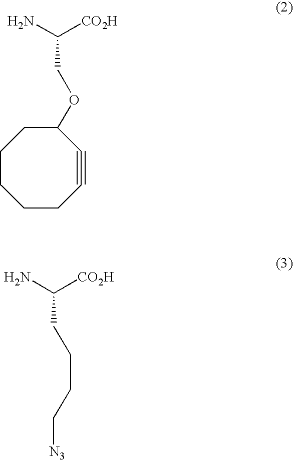

### Example 2

### Pre-Targeted Imaging of Breast Tumor Tissue for Therapy Planning

A targeting probe made up of an azide-estrogen derivative is administered to a breast cancer patient. After estrogen receptor binding, a cyclooctyne group conjugated to a ^{99m}Tc chelate is injected as an imaging probe and binds and visualizes the immobilized azide. Several breast cancer-targeted constructs functionalized with an azide are already known. None of these has been used however in a [3+2] azide-alkyne cycloaddition based imaging method.

### Example 3

### Imaging of Bone Tumour Tissue

A conjugate of a diphosphonate with a cyclooctyne group is administered as a targeting probe to a bone cancer patient. After bone accumulation, a ^{99m}Tc chelate functionalized with a pendant azide is injected into the patient as the imaging probe. Alternatively, in the context of bone cancer therapy, the imaging probe made up of the chelate-azide conjugate further carries a therapeutic nuclide. Alternatively, in the targeting probe, the diphosphonate is linked to the azide (4) and in the imaging probe, the secondary targeting group is a cyclooctyne group which is linked to the label (Tc chelate).

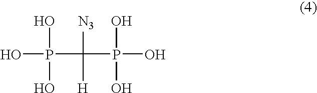

### Example 4

### Pre-Targeted Imaging of Brain Tissue

An azido-tropane derivative is injected as a targeting probe into a subject with e.g. Parkinson disease. After target binding in the dopaminergic system, an ¹⁸F-labelled cyclooctyneprobe (5) is injected as an imaging probe and binds to the immobilised azide.

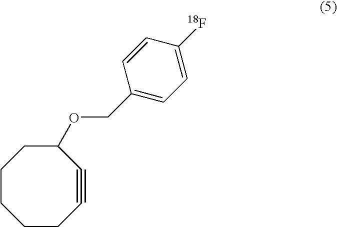

### Example 5

### Pre-Targeted Imaging of Hypoxic Tissue

Azide functionalized nitroimidazole derivatives are used as probes to image hypoxia, e.g. (6), (7) shown below. In hypoxic cells the nitro moiety is reduced to a radical, which is then trapped upon reaction with intracellular macromolecules. Subsequently, a lipophilic ¹⁸F-labelled cyclooctyne group 5 is injected, e.g. as imaging probe, to bind the accumulated azide.

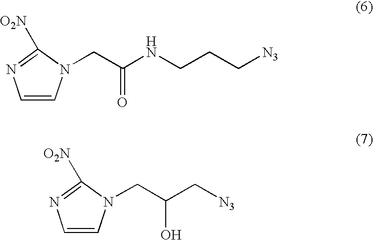

### Example 6

### Pre-Targeted Signal Amplification and/or Polyvalency Installation

A primary targeting moiety is conjugated to a dendrimer or polymer containing multiple cyclooctyne moieties. After binding of the primary targeting moiety to its primary target, e.g. a receptor, an azide conjugated to one or more MRI contrast agents, e.g. Gd chelates, or to an ultrasound reporter, e.g. microbubbles, is injected as an imaging probe. The subsequent [3+2] azide-alkyne cycloaddition results in a high concentration of MRI contrast agent at the target site. Furthermore, the polyvalency at the target site will increase the reaction kinetics with the azide reporter conjugate, affording an efficient target accumulation of MRI contrast agent or microbubbles. Alternatively, the targeting probe comprises the azide in the dendrimer and the cyclooctyne is conjugated to the label in the imaging probe.

### Example 7

### Imaging a Reporter Gene During Gene Therapy

In this application a vector is used wherein both a therapeutic gene is expressed as well as a reporter gene for the enzyme HSV1-TK. This enzyme metabolically traps uracil analogs and acycloguanosine analogs in the cell. In this embodiment, uracil and acycloguanosine analogs are functionalized with an azide moiety. These molecules are metabolically trapped in tissue where the reporter gene (and thus also the therapeutic gene) is expressed. Subsequently, an ¹⁸F-labelled cyclooctyneprobe is injected to bind the accumulated azide-comprising uracil and acycloguanosine analogs.

### Example 8

### Use of the [3+2] Azide-Alkyne Cycloaddition in the Immobilization of Gd-Dota Complexes Embedded in the Surface of Liposomes

Complex 8 with its appended stearoyl lipid residue is allowed to cover the surface of a liposome, via insertion of the stearoyl residue in the phospholipid bilayer. Next, the appended azide groups are intermolecularly cross-linked by polyfunctional cyclooctyne probe 9 (a tripeptide). The rotation of the cross linked Gd complexes will be dramatically reduced as compared to free Gd complex in the lipid, affording an increased relaxivity in MRI.

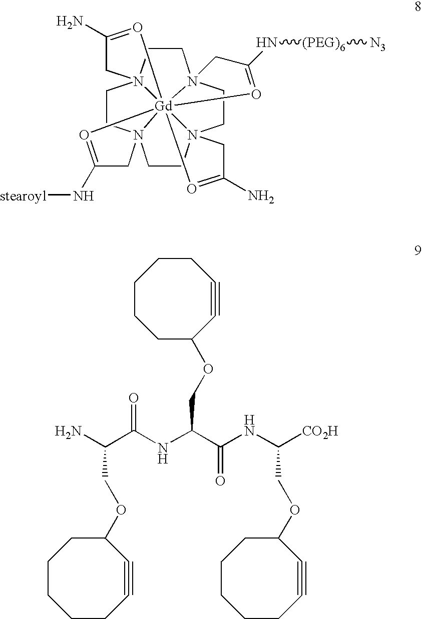

1. A kit for targeted medical imaging and/or therapeutics comprising:
at least one targeting probe comprising a primary targeting moiety and a secondary target; and at least one further probe selected from either:
an imaging probe comprising a secondary targeting moiety and a label; or
a therapeutic probe comprising a secondary targeting moiety and a pharmaceutically active compound,
characterized in that one of the targeting probe or the imaging or therapeutic probe comprises, as secondary target and secondary targeting moiety respectively, either at least one azide group and in that the other probe comprises at least one alkyne group, said alkyne and said azide groups being reaction partners for the [3+2] cycloaddition.

2. The kit according to claim 1 wherein the targeting probe comprises the at least one azide group and wherein the imaging or therapeutic probe comprises the at least one alkyne group.

3. The kit according to claim 1 wherein the alkyne group is a cycloalkyne group.

4. The kit according to claim 1 wherein the targeting probe comprises the at least one alkyne group and wherein the imaging or therapeutic probe comprises the at least one azide group.

5. The kit according to claim 1 wherein the primary targeting moiety binds to a component within the vascular system.

6. The kit according to claim 1, wherein the primary targeting moiety binds to a receptor.

7. The kit according to claim 1, wherein the primary targeting moiety binds to an intracellular component.

8. The kit according to claim 1, wherein the primary targeting moiety is an antibody.

9. The kit according to claim 1, which comprises an imaging probe.

10. The kit according to claim 9, wherein the imaging probe further comprises a pharmaceutically active compound.

11. The kit according to claim 1, which comprises a therapeutic probe.

12. An imaging probe comprising a secondary targeting moiety and a label characterized in that said imaging probe comprises as secondary targeting moiety at least one azide group or at least one alkyne group, said alkyne or said azide groups being suitable reaction partners for the [3+2] cycloaddition and in that said label is an imaging label.

13. The use of a targeting probe comprising a primary targeting moiety and a secondary target, characterized in that said targeting probe comprises as said secondary target at least one azide group or at least one alkyne, said alkyne or said azide groups being suitable reaction partners for the [3+2] cycloaddition, as a tool in targeted medical imaging.

14. The use of a targeting probe comprising a primary targeting moiety and a secondary target, characterized in that said targeting probe comprises as said secondary target at least one azide group or at least one alkyne group, said alkyne or said azide groups being suitable reaction partners for the [3+2] cycloaddition, in the manufacture of a tool for medical imaging.

15. A combined probe for medical imaging comprising a primary targeting moiety and a detectable label characterized in that the targeting moiety is connected to the detectable label via a triazole adduct.

16. A method of in vitro preparing a combined targeting and imaging or therapeutic probe, comprising a primary targeting moiety and a detectable label or a pharmaceutically active agent, comprising the step of reacting an alkyne comprising detectable label with an azide-comprising primary targeting moiety or reacting an azide-comprising detectable label with an alkyne-comprising primary targeting moiety.

17. A method of developing a targeting probe with optimal binding affinity for a target and optimal reaction with an imaging or therapeutic probe, which comprises:
a) making a compound library of the targeting moiety of said targeting probe, whereby the secondary target is introduced at different sites on said targeting moiety;
b) screening the so obtained compound library for binding with the target and with an imaging and/or targeting probe.

18. A kit for targeted medical imaging and/or therapeutics comprising:
at least one target metabolic precursor comprising a secondary target; and at least one further probe selected from either:
an imaging probe comprising a secondary targeting moiety and a label; or
a therapeutic probe comprising a secondary targeting moiety and a pharmaceutically active compound,
characterized in that one of the target metabolic substrate or the imaging or therapeutic probe comprises, as secondary target and secondary targeting moiety respectively, either at least one azide group and in that the other probe comprises at least one alkyne group, said alkyne and said azide groups being reaction partners for the [3+2] cycloaddition.

19. A kit for targeted medical imaging and/or therapeutics comprising:
at least one reporter probe comprising a secondary target; and at least one further probe selected from either:
an imaging probe comprising a secondary targeting moiety and a label; or
a therapeutic probe comprising a secondary targeting moiety and a pharmaceutically active compound,
characterized in that one of the reporter or the imaging or therapeutic probe comprises, as secondary target and secondary targeting moiety respectively, either at least one azide group and in that the other probe comprises at least one cycloalkyne group, said alkyne and said azide groups being reaction partners for the [3+2] cycloaddition.
